(19) United States
(12) Patent Application Publication
(10) Pub. No.: US 2025/0226070 A1
(43) Pub. Date: Jul. 10, 2025

(54) IDENTIFICATION OF DIRECT EFFECT MODIFIERS (DEM) USING ITERATIVE ORTHOGONAL REGRESSION (IOR) FOR HETEROGENEOUS EFFECT ASSESSMENT

(71) Applicant: Covera Health, New York, NY (US)

(72) Inventors: James A. Belmonte, Landisville, PA (US); Nikola Andric, Watchung, NJ (US); Ryan M McKenna, Philadelphia, PA (US); Benjamin L Odry, West New York, NJ (US); Ron Vianu, New York, NY (US)

(21) Appl. No.: 19/016,266

(22) Filed: Jan. 10, 2025

Related U.S. Application Data
(60) Provisional application No. 63/619,391, filed on Jan. 10, 2024.

(51) Int. Cl. G16H 20/00; G16H 50/50; G16H 50/70

(52) U.S. Cl. CPC G16H 20/00; G16H 50/50; G16H 50/70

(57) ABSTRACT

A process for determining direct effect modifiers (DEMs) for an exposure of interest can include determining pre-treatment variables corresponding to characteristics of different subsets of an observed population for the exposure of interest, each individual of the observed population having a respective exposure to the exposure of interest and a corresponding outcome. A predicted conditional average treatment effect (CATE) can be determined for the exposure of interest on the observed population. Each variable in a matrix of covariates selected from the pre-treatment variables can be orthogonalized to the remaining covariates. Regression can be performed on the predicted CATE to determine additional residuals for each pre-treatment variable. Variables with additional residuals significantly associated with CATE are inferred to be DEMs.

### CROSS-REFERENCE TO RELATED APPLICATIONS

This application claims the benefit of priority to U.S. Provisional Patent Application No. 63/619,391, filed Jan. 10, 2024, and entitled “BEYOND CONDITIONAL AVERAGE TREATMENT EFFECT (CATE) PREDICTION, IDENTIFICATION OF DIRECT MODIFIERS USING ITERATIVE ORTHOGONAL REGRESSION,” which is hereby incorporated by reference, in its entirety and for all purposes.

### TECHNICAL FIELD

The present disclosure relates generally to causal inference, and more specifically pertains to systems and techniques for a framework to identify causal (e.g., direct) effect modifiers of a given treatment or other binary, categorical, or continuous exposure(s) of interest.

### BACKGROUND

Estimating the causal effects of a treatment or exposure type (e.g., binary, categorical, continuous, etc.) from observations is an important task in various fields where there is a need to understand the differential impact of potential interventions or treatments that may be provided for different subpopulations of an overall population. For instance, causal effect estimation can be utilized in fields such as medicine, econometrics, and public policy, among various others. In the field of causal inference, conditional average treatment effects (CATE) can be used to characterize the heterogeneity in treatment effects across different subpopulations within a broader population. For example, CATE estimation can be performed to determine the average effect of a treatment for individuals with specific characteristics or within defined subgroups of a broader, observed population undergoing the binary treatment under study. The specific characteristics, subgroups, and/or subpopulations can be represented as respective covariates upon which the CATE is conditioned. For instance, in some examples, CATE may be defined as a difference of the expected values of binary treatments' scalar outcomes conditioned on covariates, where different covariates correspond to different subpopulations within the broader population of the observations.

More particularly, in the binary treatment case, CATE estimation may be performed based an assumption of potential outcome pairs corresponding to a treatment, wherein each individual of a population has a first outcome if receiving the treatment and a second outcome if not receiving the treatment. The treatment effect for a given individual is the difference between the two potential outcomes; CATE is the expected difference in potential outcomes given a set of covariates which characterize a subpopulation of interest. A fundamental challenge of causal inference is that, in real-world scenarios, the true pair of potential outcomes cannot be fully observable for a single individual, as an individual can only receive the treatment or not receive the treatment (e.g., if receiving the treatment, the individual's outcome when not receiving the treatment is unobservable; if not receiving the treatment, the individual's outcome when receiving the treatment is unobservable).

CATE can be estimated for binary, categorical, and/or continuous treatments.

Various techniques and/or methodologies exist for performing CATE estimation. For instance, in some examples, CATE can be determined or otherwise estimated using a fully parametric approach (e.g., using a fully parametric model for the potential outcomes). A fully parametric approach to CATE estimation can be based on specifying a parametric model that represents the relationships between the treatment, the outcome, and the covariates. A challenge in the fully parametric approach to CATE estimation is that the true, underlying form of the relationship(s) between the outcome, treatment, and covariates is often unknown, and such an approach therefore risks model misspecification when the true relationships deviate from the assumed functional form used in the parametric model.

Advances in causal machine learning have provided techniques to non-parametrically predict CATE, thus facilitating effect modifier identification through various techniques for testing the associations between variables and estimated CATE. Machine learning-based techniques for estimating or predicting CATE may achieve an accuracy that is better than, the same as, or worse than an accuracy associated with the fully parametric approach. However, even with a perfect CATE estimation, the machine learning-based techniques cannot differentiate between different types or degrees of effect modifiers for the CATE. For example, the machine learning-based techniques cannot differentiate between effect modifiers that cause effect heterogeneity (e.g., direct effect modifiers or “DEMs”) and effect modifiers that are simply associated with effect heterogeneity (e.g., non-DEMs, such as indirect effect modifiers, common cause effect modifiers, and/or proxy effect modifiers).

### SUMMARY

The accompanying drawings are presented to aid in the description of various aspects of the disclosure and are provided solely for illustration of the aspects and not limitation thereof.

In one illustrative example, a method is provided for determining one or more direct effect modifiers for an exposure of interest, the method comprising: determining a plurality of pre-treatment variables corresponding to characteristics of different subsets of an observed population for the exposure of interest, wherein the observed population comprises a plurality of individuals each having a respective exposure to the exposure of interest and a corresponding outcome; generating a predicted conditional average treatment effect (CATE) information for the exposure of interest on the observed population, wherein the predicted CATE information is generated by a causal machine learning model configured with a covariate matrix of a set of variables comprising at least a portion of the plurality of pre-treatment variables; for each respective variable included in the set of variables of the covariate matrix: fitting a configured model using a remaining set of variables with the respective variable removed, and determining a residual representation for the respective variable based on the fitting, wherein the residual representation for the respective variable is orthogonal to each variable in the remaining set of variables; regressing the predicted CATE information on the residual representation for each respective variable to thereby determine additional residuals for each regressed residual representation; determining association significance information between the CATE and the additional residuals of each respective variable; and outputting an indication of the one or more direct effect modifiers (DEMs) for the exposure of interest, wherein the one or more direct effect modifiers are the respective variables of the covariate matrix having association significance above a threshold value with the CATE.

In some aspects, the method further comprises: calculating p-values for the additional residuals of each respective variable with the CATE; and identifying the one or more DEMs for the exposure of interest as the pre-treatment variables with calculated p-values above a configured threshold value of significance.

In some aspects, the calculated p-values and the association significance information are the same.

In some aspects, determining the association significance information between the CATE and the additional residuals of each respective variable comprises testing at least one of: linear association or polynomial associations between the CATE and the additional residuals.

In some aspects, determining the residual representation for the respective variable based on the fitting of the model comprises a first step performed by an iterative orthogonal regression (IOR) engine, and wherein regressing the predicted CATE information comprises a second step performed by the IOR engine.

In some aspects, the corresponding outcome for each individual of the observed population comprises a treatment effect of the individual's respective exposure to the exposure of interest.

In some aspects, the one or more DEMs are identified from the plurality of pre-treatment variables as a respective one or more pre-treatment variables inferred to cause effect heterogeneity in the outcome for each individual's exposure to the exposure of interest.

In some aspects, the method further comprises outputting an indication of one or more pre-treatment variables identified from the plurality of pre-treatment variables as non-DEMs, wherein each non-DEM is associated with but does not cause the effect heterogeneity.

In some aspects, the exposure comprises a binary exposure, a categorical exposure, or a continuous exposure.

In another illustrative example, provided is an apparatus comprising: at least one memory; and at least one processor coupled to the at least one memory, the at least one processor configured to: determine a plurality of pre-treatment variables corresponding to characteristics of different subsets of an observed population for an exposure of interest, wherein the observed population comprises a plurality of individuals each having a respective exposure to the exposure of interest and a corresponding outcome; generate a predicted conditional average treatment effect (CATE) information for the exposure of interest on the observed population, wherein the predicted CATE information is generated by a causal machine learning model configured with a covariate matrix of a set of variables comprising at least a portion of the plurality of pre-treatment variables; for each respective variable included in the set of variables of the covariate matrix: fit a configured model using a remaining set of variables with the respective variable removed, and determine a residual representation for the respective variable based on the fitting, wherein the residual representation for the respective variable is orthogonal to each variable in the remaining set of variables; regress the predicted CATE information on the residual representation for each respective variable to thereby determine additional residuals for each regressed residual representation; determine association significance information between the CATE and the additional residuals of each respective variable; and output an indication of one or more direct effect modifiers (DEMs) for the exposure of interest, wherein the one or more direct effect modifiers are the respective variables of the covariate matrix having association significance above a threshold value with the CATE.

In some aspects, the at least one processor is further configured to: calculate p-values for the additional residuals of each respective variable with the CATE; and identify the one or more DEMs for the exposure of interest as the pre-treatment variables with calculated p-values above a configured threshold value of significance.

In some aspects, the calculated p-values and the association significance information are the same.

In some aspects, determining the association significance information between the CATE and the additional residuals of each respective variable comprises testing at least one of: linear association or polynomial associations between the CATE and the additional residuals.

In some aspects, determining the residual representation for the respective variable based on the fitting of the model comprises a first step performed by an iterative orthogonal regression (IOR) engine, and wherein regressing the predicted CATE information comprises a second step performed by the IOR engine.

In some aspects, the corresponding outcome for each individual of the observed population comprises a treatment effect of the individual's respective exposure to the exposure of interest.

In some aspects, the one or more DEMs are identified from the plurality of pre-treatment variables as a respective one or more pre-treatment variables inferred to cause effect heterogeneity in the outcome for each individual's exposure to the exposure of interest.

In some aspects, the at least one processor is further configured to output an indication of one or more pre-treatment variables identified from the plurality of pre-treatment variables as non-DEMs, wherein each non-DEM is associated with but does not cause the effect heterogeneity.

In some aspects, the exposure comprises a binary exposure, a categorical exposure, or a continuous exposure.

In another illustrative example, provided is a non-transitory computer-readable storage medium having stored therein instructions which, when executed by a processor, cause the processor to perform operations comprising: determining a plurality of pre-treatment variables corresponding to characteristics of different subsets of an observed population for an exposure of interest, wherein the observed population comprises a plurality of individuals each having a respective exposure to the exposure of interest and a corresponding outcome; generating a predicted conditional average treatment effect (CATE) information for the exposure of interest on the observed population, wherein the predicted CATE information is generated by a causal machine learning model configured with a covariate matrix of a set of variables comprising at least a portion of the plurality of pre-treatment variables; for each respective variable included in the set of variables of the covariate matrix: fitting a configured model using a remaining set of variables with the respective variable removed, and determining a residual representation for the respective variable based on the fitting, wherein the residual representation for the respective variable is orthogonal to each variable in the remaining set of variables; regressing the predicted CATE information on the residual representation for each respective variable to thereby determine additional residuals for each regressed residual representation; determining association significance information between the CATE and the additional residuals of each respective variable; and outputting an indication of one or more direct effect modifiers (DEMs) for the exposure of interest, wherein the one or more direct effect modifiers are the respective variables of the covariate matrix having association significance above a threshold value with the CATE.

In some aspects, the instructions further cause the processor to perform operations comprising: calculating p-values for the additional residuals of each respective variable with the CATE; and identifying the one or more DEMs for the exposure of interest as the pre-treatment variables with calculated p-values above a configured threshold value of significance.

### BRIEF DESCRIPTION OF THE DRAWINGS

The accompanying drawings are presented to aid in the description of various aspects of the disclosure and are provided solely for illustration of the aspects and not limitation thereof.

FIG. 1 illustrates an example implementation of a System-on-a-Chip (SoC), in accordance with some examples;

FIG. 2A and FIG. 2B illustrate an example of a fully connected neural network, in accordance with some examples;

FIG. 3A is a directed acyclic graph (DAG) representing the causal structure between different variables and an outcome and exposure, in accordance with some examples;

FIG. 3B is a DAG representing the causal structure between the variables of FIG. 3A and the treatment effect of the exposure, in accordance with some examples;

FIG. 4 is a diagram illustrating an example of a causal directed acyclic graph (DAG) depicting different types of effect modifiers and their respective causal structures as they relate to a Conditional Average Treatment Effect (CATE), in accordance with some examples;

FIG. 5 is an example of a causal DAG corresponding to a Common Cause effect modifier that may be associated with but does not directly modify the treatment effect or cause effect heterogeneity, in accordance with some examples;

FIG. 6 is an example of a causal DAG corresponding to an Indirect effect modifier that may be associated with but does not directly modify the treatment effect or cause effect heterogeneity, in accordance with some examples;

FIG. 7 is an example of a causal DAG corresponding to a Direct effect modifier (DEM) that directly modifies the treatment effect and/or causes effect heterogeneity, in accordance with some examples;

FIG. 8 is an example of a causal DAG corresponding to a Proxy effect modifier that may be associated with but does not directly modify the treatment effect or cause effect heterogeneity, in accordance with some examples; and

FIG. 9 illustrates an example computing system that can be used to implement various aspects described herein.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure. Additional features and advantages of the disclosure will be set forth in the description which follows, and in part will be obvious from the description, or can be learned by practice of the herein disclosed principles. It will be appreciated that for simplicity and clarity of illustration, where appropriate, reference numerals have been repeated among the different figures to indicate corresponding or analogous elements. The description is not to be considered as limiting the scope of the embodiments described herein.

### Overview

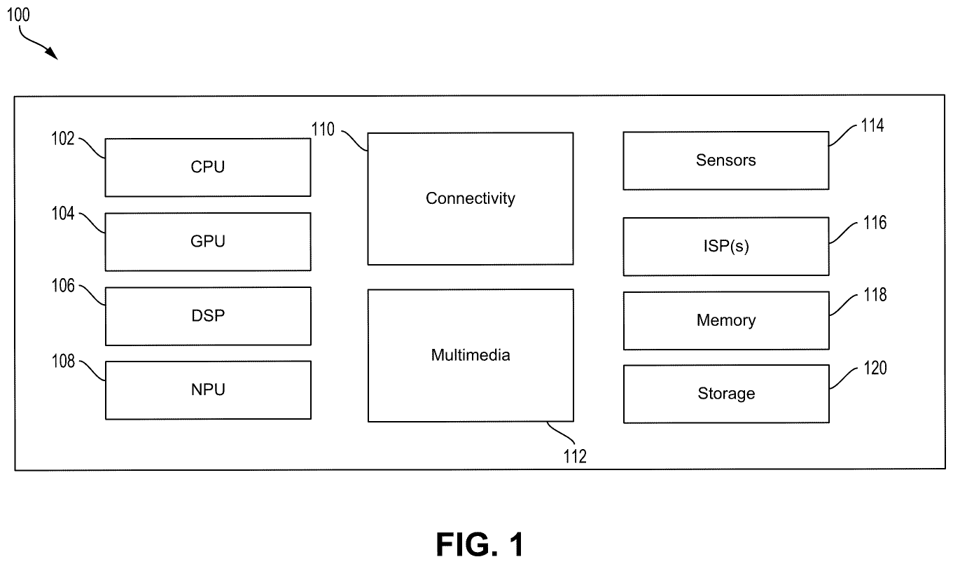

FIG. 1 illustrates an example implementation of a system-on-a-chip (SoC) 100, which may include a central processing unit (CPU) 102 or a multi-core CPU, configured to perform one or more of the functions described herein. Parameters or variables (e.g., neural signals and synaptic weights), system parameters associated with a computational device (e.g., neural network with weights), delays, frequency bin information, task information, among other information may be stored in a memory block associated with a neural processing unit (NPU) 108, in a memory block associated with a CPU 102, in a memory block associated with a graphics processing unit (GPU) 104, in a memory block associated with a digital signal processor (DSP) 106, in a memory block 118, and/or may be distributed across multiple blocks. Instructions executed at the CPU 102 may be loaded from a program memory associated with the CPU 102 or may be loaded from a memory block 118. The SoC 100 may also include additional processing blocks tailored to specific functions, such as a GPU 104, a DSP 106, a connectivity block 110, which may include fifth generation (5G) connectivity, fourth generation long term evolution (4G LTE) connectivity, Wi-Fi connectivity, USB connectivity, Bluetooth connectivity, and the like, and a multimedia processor 112 that may, for example, detect and recognize gestures, speech, and/or other interactive user action(s) or input(s). In one implementation, the NPU is implemented in the CPU 102, DSP 106, and/or GPU 104. The SoC 100 may also include a sensor processor 114, image signal processors (ISPs) 116, and/or storage 120. In some examples, the sensor processor 114 can be associated with or connected to one or more sensors for providing sensor input(s) to sensor processor 114. For example, the one or more sensors and the sensor processor 114 can be provided in, coupled to, or otherwise associated with a same computing device. The SoC 100 may be based on an ARM instruction set. In an aspect of the present disclosure, the instructions loaded into the CPU 102 may comprise code to search for a stored multiplication result in a lookup table (LUT) corresponding to a multiplication product of an input value and a filter weight. The instructions loaded into the CPU 102 may also comprise code to disable a multiplier during a multiplication operation of the multiplication product when a lookup table hit of the multiplication product is detected. In addition, the instructions loaded into the CPU 102 may comprise code to store a computed multiplication product of the input value and the filter weight when a lookup table miss of the multiplication product is detected.

In some implementations, the CPU 102, the GPU 104, the DSP 106, the NPU 108, the connectivity block 110, the multimedia processor 112, the one or more sensors 114, the ISPs 116, the memory block 118 and/or the storage 120 can be part of the same computing device. For example, in some cases, the CPU 102, the GPU 104, the DSP 106, the NPU 108, the connectivity block 110, the multimedia processor 112, the one or more sensors 114, the ISPs 116, the memory block 118 and/or the storage 120 can be integrated into a smartphone, laptop, tablet computer, smart wearable device, video gaming system, server, and/or any other computing device. In other implementations, the CPU 102, the GPU 104, the DSP 106, the NPU 108, the connectivity block 110, the multimedia processor 112, the one or more sensors 114, the ISPs 116, the memory block 118 and/or the storage 120 can be part of two or more separate computing devices.

Machine learning (ML) can be considered a subset of artificial intelligence (AI). ML systems can include algorithms and statistical models that computer systems can use to perform various tasks by relying on patterns and inference, without the use of explicit instructions. One example of a ML system is a neural network (also referred to as an artificial neural network), which may include an interconnected group of artificial neurons (e.g., neuron models). Neural networks may be used for various applications and/or devices, such as speech analysis, audio signal analysis, image and/or video coding, image analysis and/or computer vision applications, Internet Protocol (IP) cameras, Internet of Things (IoT) devices, autonomous vehicles, service robots, among others.

Individual nodes in a neural network may emulate biological neurons by taking input data and performing simple operations on the data. The results of the simple operations performed on the input data are selectively passed on to other neurons. Weight values are associated with each vector and node in the network, and these values constrain how input data is related to output data. For example, the input data of each node may be multiplied by a corresponding weight value, and the products may be summed. The sum of the products may be adjusted by an optional bias, and an activation function may be applied to the result, yielding the node's output signal or “output activation” (sometimes referred to as a feature map or an activation map). The weight values may initially be determined by an iterative flow of training data through the network (e.g., weight values are established during a training phase in which the network learns how to identify particular classes by their typical input data characteristics).

Different types of neural networks exist, such as convolutional neural networks (CNNs), recurrent neural networks (RNNs), generative adversarial networks (GANs), multilayer perceptron (MLP) neural networks, transformer neural networks, among others. For instance, convolutional neural networks (CNNs) are a type of feed-forward artificial neural network. Convolutional neural networks may include collections of artificial neurons that each have a receptive field (e.g., a spatially localized region of an input space) and that collectively tile an input space. RNNs work on the principle of saving the output of a layer and feeding this output back to the input to help in predicting an outcome of the layer. A GAN is a form of generative neural network that can learn patterns in input data so that the neural network model can generate new synthetic outputs that reasonably could have been from the original dataset. A GAN can include two neural networks that operate together, including a generative neural network that generates a synthesized output and a discriminative neural network that evaluates the output for authenticity. In MLP neural networks, data may be fed into an input layer, and one or more hidden layers provide levels of abstraction to the data. Predictions may then be made on an output layer based on the abstracted data.

Deep learning (DL) is one example of a machine learning technique and can be considered a subset of ML. Many DL approaches are based on a neural network, such as an RNN or a CNN, and utilize multiple layers. The use of multiple layers in deep neural networks can permit progressively higher-level features to be extracted from a given input of raw data. For example, the output of a first layer of artificial neurons becomes an input to a second layer of artificial neurons, the output of a second layer of artificial neurons becomes an input to a third layer of artificial neurons, and so on. Layers that are located between the input and output of the overall deep neural network are often referred to as hidden layers. The hidden layers learn (e.g., are trained) to transform an intermediate input from a preceding layer into a slightly more abstract and composite representation that can be provided to a subsequent layer, until a final or desired representation is obtained as the final output of the deep neural network.

As noted above, a neural network is an example of a machine learning system, and can include an input layer, one or more hidden layers, and an output layer. Data is provided from input nodes of the input layer, processing is performed by hidden nodes of the one or more hidden layers, and an output is produced through output nodes of the output layer. Deep learning networks typically include multiple hidden layers. Each layer of the neural network can include feature maps or activation maps that can include artificial neurons (or nodes). A feature map can include a filter, a kernel, or the like. The nodes can include one or more weights used to indicate an importance of the nodes of one or more of the layers. In some cases, a deep learning network can have a series of many hidden layers, with early layers being used to determine simple and low-level characteristics of an input, and later layers building up a hierarchy of more complex and abstract characteristics.

A deep learning architecture may learn a hierarchy of features. If presented with visual data, for example, the first layer may learn to recognize relatively simple features, such as edges, in the input stream. In another example, if presented with auditory data, the first layer may learn to recognize spectral power in specific frequencies. The second layer, taking the output of the first layer as input, may learn to recognize combinations of features, such as simple shapes for visual data or combinations of sounds for auditory data. For instance, higher layers may learn to represent complex shapes in visual data or words in auditory data. Still higher layers may learn to recognize common visual objects or spoken phrases. Deep learning architectures may perform especially well when applied to problems that have a natural hierarchical structure. For example, the classification of motorized vehicles may benefit from first learning to recognize wheels, windshields, and other features. These features may be combined at higher layers in different ways to recognize cars, trucks, and airplanes. Neural networks may be designed with a variety of connectivity patterns. In feed-forward networks, information is passed from lower to higher layers, with each neuron in a given layer communicating to neurons in higher layers. A hierarchical representation may be built up in successive layers of a feed-forward network, as described above. Neural networks may also have recurrent or feedback (also called top-down) connections. In a recurrent connection, the output from a neuron in a given layer may be communicated to another neuron in the same layer. A recurrent architecture may be helpful in recognizing patterns that span more than one of the input data chunks that are delivered to the neural network in a sequence. A connection from a neuron in a given layer to a neuron in a lower layer is called a feedback (or top-down) connection. A network with many feedback connections may be helpful when the recognition of a high-level concept may aid in discriminating the particular low-level features of an input.

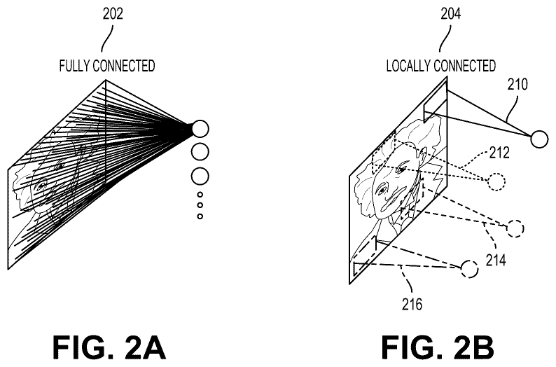

The connections between layers of a neural network may be fully connected or locally connected. FIG. 2A illustrates an example of a fully connected neural network 202. In a fully connected neural network 202, a neuron in a first layer may communicate its output to every neuron in a second layer, so that each neuron in the second layer will receive input from every neuron in the first layer. FIG. 2B illustrates an example of a locally connected neural network 204. In a locally connected neural network 204, a neuron in a first layer may be connected to a limited number of neurons in the second layer. More generally, a locally connected layer of the locally connected neural network 204 may be configured so that each neuron in a layer will have the same or a similar connectivity pattern, but with connections strengths that may have different values (e.g., 210, 212, 214, and 216). The locally connected connectivity pattern may give rise to spatially distinct receptive fields in a higher layer, as the higher layer neurons in a given region may receive inputs that are tuned through training to the properties of a restricted portion of the total input to the network.

### Example Embodiments

Described herein are systems and techniques that can be used to identify or otherwise determine one or more direct effect modifiers for an exposure of interest, including in clinical and/or medical contexts, scenarios, examples, etc. For example, the systems and techniques can be used to differentiate between associative and causal effect modifiers. An effect modifier may be a variable or influencing factor that influences the magnitude and/or direction of the effect of an exposure (e.g., a treatment, an intervention, etc.) on a given outcome. In a simple example of a health intervention, factors such as age may be seen as effect modifiers, on the basis that such factors (e.g., age) might alter the effectiveness of a treatment included in the health intervention, etc.

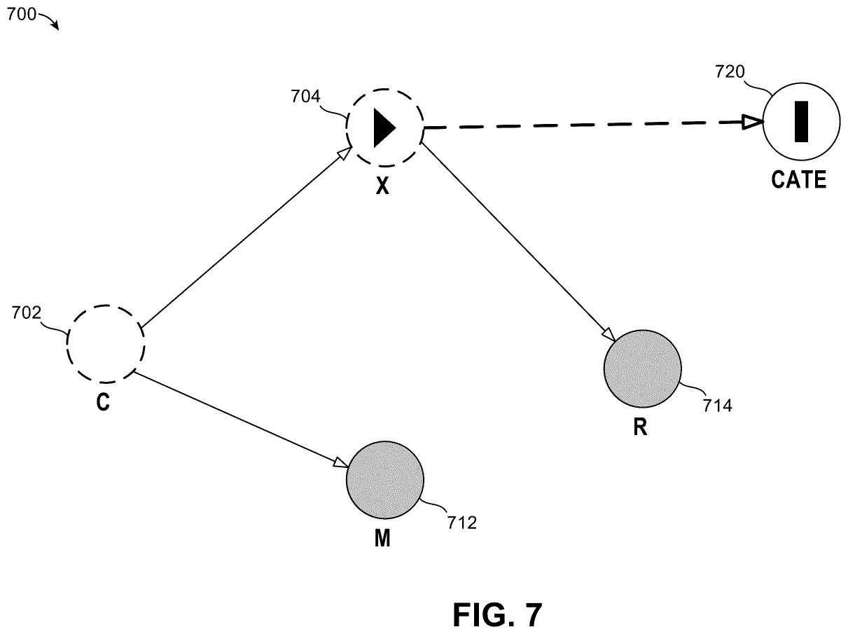

As referred to herein, a direct effect modifier (DEM) can refer to an effect modifier that causes effect heterogeneity. A DEM may also be referred to as a causal effect modifier. In the context of the present disclosure, an illustrative example of a direct effect modifier (DEM or causal effect modifier) is described below with respect to at least the causal directed acyclic graph (DAG) of FIG. 7. A direct effect modifier may be identified as directly influencing a treatment effect, and is not blocked by other variables. A DEM can therefore be understood to cause genuine effect heterogeneity.

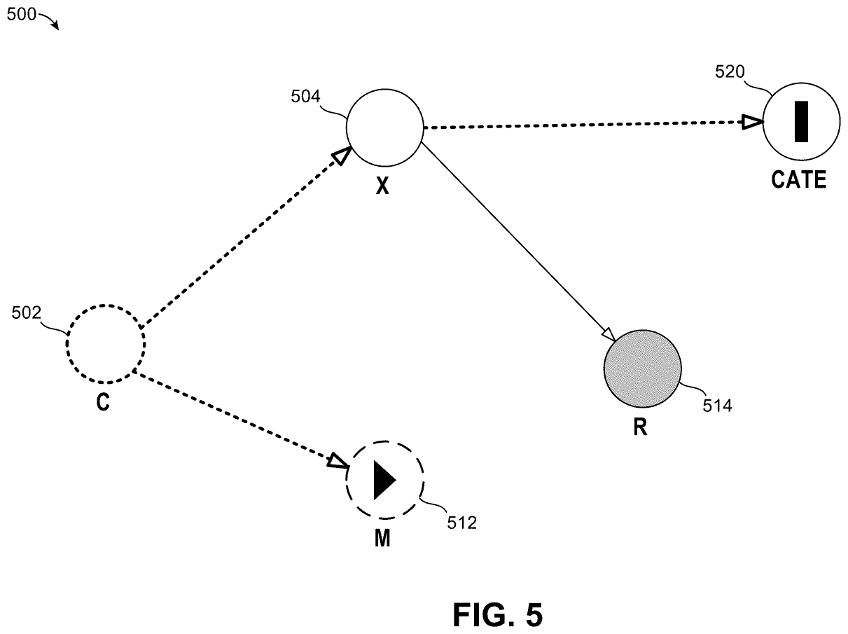

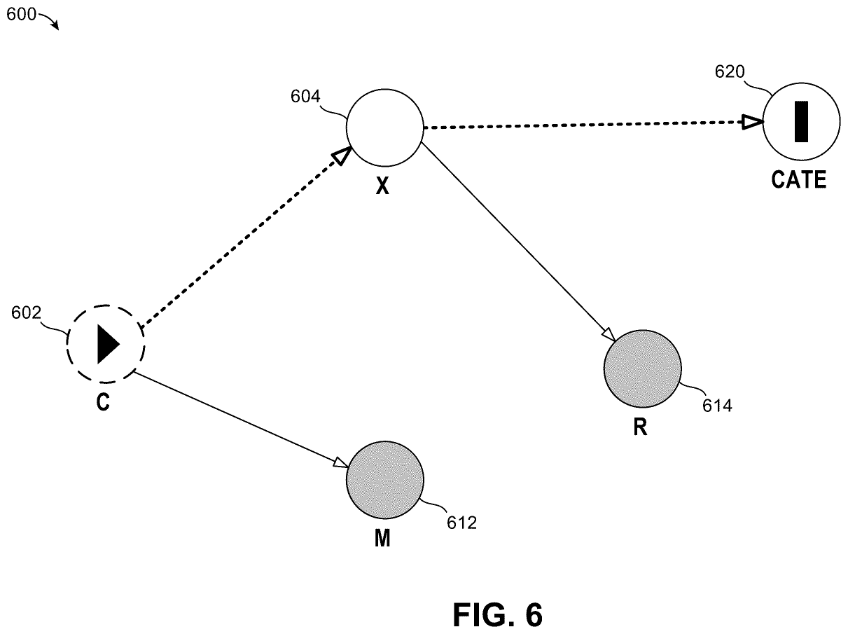

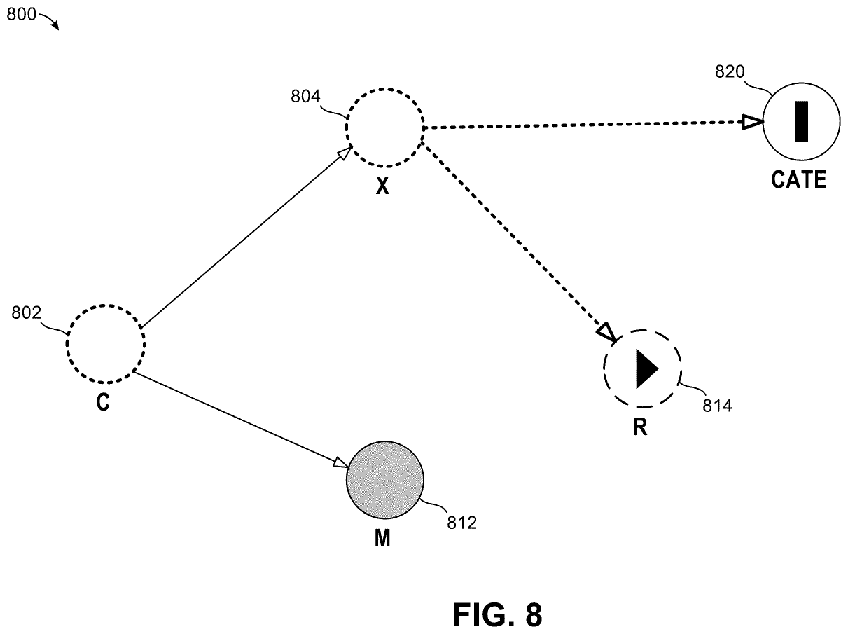

An effect modifier that is not a causal (direct) effect modifier can be referred to as a non-DEM and/or an associative effect modifier. As such, compared to a direct (causal) effect modifier, non-DEM (e.g., associative) effect modifiers may be associated with differences in a treatment effect, without rising to the level of directly causing the difference(s). For example, the differences in treatment effect that are associated with non-DEM (associative) effect modifiers may have relationships that are explained by other variables or pathways. In some cases, a non-DEM is an effect modifier that is associated with, but does not cause, effect heterogeneity. In one illustrative example, non-DEM (associative) effect modifier types can be characterized into different sub-categories or sub-types. For example, a first type of non-DEM/associative effect modifier type can be referred to as Common Cause effect modifiers, an example of which is described with respect to at least the causal DAG of FIG. 5. A second type of non-DEM/associative effect modifier type can be referred to as Indirect effect modifiers, an example of which is described with respect to at least the causal DAG of FIG. 6. A third type of non-DEM/associative effect modifier type can be referred to as Proxy effect modifiers, an example of which is described with respect to at least the causal DAG of FIG. 8.

The systems and techniques described herein can be used for the identification of DEMs, based on the presently disclosed framework for Iterative Orthogonal Regression (IOR). In some examples, the systems and techniques can be used for the identification of DEMs using IOR for heterogeneous effect assessment. As will be described in greater depth below, iterative orthogonal regression can be used to differentiate between effect modifiers that cause effect heterogeneity (e.g., DEMs/causal effect modifiers) and effect modifiers that are only associated with, but do not cause, effect heterogeneity (e.g., non-DEMs/associative effect modifiers, such as common cause, indirect, and/or proxy effect modifiers). In one illustrative example, one or more DEMs can be identified from a plurality of effect modifiers associated with a CATE estimation, wherein the identification of the DEMs is based on iteratively testing the association between the residuals of each respective effect modifier and the estimated CATE. In some aspects, each effect modifier is residualized by the remaining effect modifiers prior to iteratively testing the associations, as will be described in greater depth below.

Notably, based on determining one or more DEMs (e.g., determining one or more DEMs from a plurality of effect modifiers for a treatment or intervention, etc.), the systems and techniques can be used to further enable the determination of which patient features are causing differential response to the treatment or intervention under study. Even with perfect CATE estimation, the CATE alone does not provide sufficient information to test for associations between variables and the treatment effect in order to distinguish between causal and associative effect modifiers. The systems and techniques described herein can implement the disclosed iterative orthogonal regression (IOR) framework to identify causal (direct) effect modifiers for binary, categorical, and/or continuous exposures. The IOR framework disclosed herein can additionally be used for various example tasks and implementations, including but not limited to examples such as uncovering causal mechanisms, researching health equity, and/or program optimization, etc., among various others.

### Conditional Average Treatment Effect (CATE)

As noted previously, in the field of causal inference, conditional average treatment effect (CATE) can be used to characterize the heterogeneity in treatment effects across different subpopulations within a broader population. For example, CATE estimation can be performed to determine the average effect of a treatment for individuals with specific characteristics or within defined subgroups of a broader, observed population undergoing the binary treatment under study. The specific characteristics, subgroups, and/or subpopulations can be represented as respective covariates upon which the CATE is conditioned. For instance, in some examples, CATE may be defined as a difference of the expected values of binary treatments' scalar outcomes conditioned on covariates, where different covariates correspond to different subpopulations within the broader population of the observations.

In one illustrative example, CATE can be given as:

$$\hat{τ} ( X ) = E [ Y ( W ) - Y ( {W}^{*} ) \vert X ] \qquad Eq . ( 1 )$$

Here, the term X represents a matrix of pre-treatment variables (e.g., covariates). The function Y ( ) represents the outcome of interest under a particular one of a binary treatment status pair W or W*. For example, W may represent receiving the treatment while W* represents not receiving the treatment (e.g., W is the active treatment and W* is the control treatment). The term \hat{τ} (x) is a vector of the predicted CATE. As contemplated herein, CATE can be estimated using any applicable methodology, technique, etc., (including machine learning-based methodologies for CATE estimation) for binary, categorical, or continuous treatments.

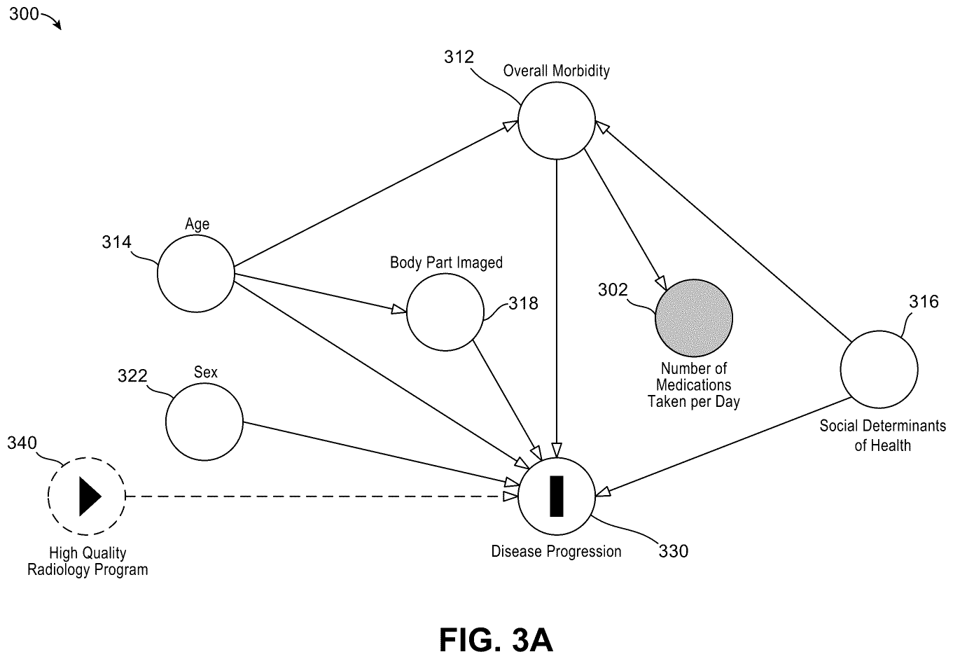

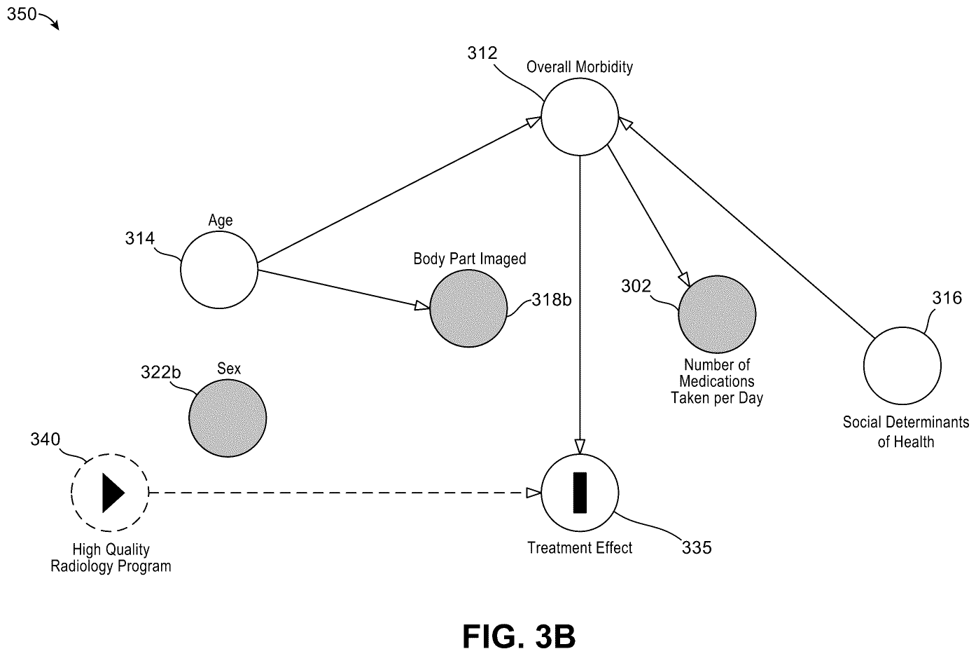

FIG. 3A is a diagram illustrating an example of a causal directed acyclic graph (DAG) 300, where the causal DAG 300 corresponds to an illustrative example of a scenario where a particular high-quality radiology program is an exposure of interest and the effect of the exposure on patient disease progression is being measured. In some embodiments, the causal DAG 300 corresponds to an example scenario where patient disease progression is measured with a particular high-quality radiology program as the exposure of interest, with the causal DAG 300 representative of the causal structure between different variables, the outcome, and the exposure. In particular, the causal DAG 300 represents a causal structure between an outcome 330 given as “Disease Progression,” an exposure 340 given as “High Quality Radiology Program,” and the various different variables 312 (e.g., “Overall Morbidity”), 314 (e.g., “Age”), 318 (e.g., “Body Part Imaged”), 302 (e.g., “Number of Medications Taken per Day”), 316 (e.g., “Social Determinants of Health”), and 322 (e.g., “Sex”).

In the example(s) of FIGS. 3A-8, an outcome under study/consideration is depicted as a circle containing a vertical black bar and having a solid line circumference (e.g., such as the outcome 330 of FIG. 3A, etc.). An exposure under study/consideration, and associated with the outcome, is depicted as a circle containing a black triangle and having a dashed line circumference (e.g., such as the exposure 340 of FIG. 3A, etc.). Variables are depicted as solid line circumference circles having either a white fill or a gray fill (e.g., such as the variables 302, 312, 314, 316, 318, 322 of FIG. 3A, etc.).

In the example causal DAG 300 of FIG. 3A, the exposure 330 of an observed population of patients to the high-quality radiology program 340 is randomized. For example, a given patient included in the observed population of patients either receives treatment under the high-quality radiology program 340 (e.g., treatment status W of Eq. (1)) or does not receive treatment under the high quality radiology program 340 (e.g., treatment status W* of Eq. (1)). The patients that do not receive treatment under the high quality radiology program 340 can comprise patients that receive the control treatment for the study.

Because the exposure 340 is randomized for each patient in the observed population, there accordingly are no nodes in the causal DAG 300 that have arrows terminating (e.g., indicating a relationship that may or may not be causal, may or may not be associative, etc.) towards the “High Quality Radiology Program” exposure node 340 of the causal DAG 300. In other words, nothing causes the exposure 340 except for a random assignment.

In the context of the causal DAG 300, the outcome is represented as the “Disease Progression” node 330, as noted above. The outcome 330 is caused by the set of variables that are connected to the outcome 330 node in the causal DAG 300, where each variable is a characteristic of the patient prior to being imaged under either the active treatment of “High Quality Radiology” (e.g., treatment status W) or the control treatment of standard radiology (e.g., treatment status W*). For example, the outcome 330 is caused by the set of variables corresponding to and/or associated with the Age node 314, the Overall Morbidity node 312, the Body Part Imaged node 318, the Social Determinants of Health (SDOH) node 316, and the Sex node 322.

The five variables above are characteristics of the patient that cause the Disease Progression outcome 330. The causal relationship is represented by the solid white fill and solid line circumferences of each respective node associated with a variable that causes the outcome 330 of “Disease Progression.” An additional node 302 corresponds to a sixth variable, “# Of Medications Taken Per Day”. It is noted that the medications node 302 is depicted with a gray fill, rather than a white fill like the other node variables, indicative of the fact that the number of medications taken per day (e.g., the variable of node 302) is caused by the “Overall Morbidity” node 312, but does not itself directly cause the outcome 330 of “Disease Progression.” Accordingly, the “# Of Medications Taken Per Day” node 302 is shown as a gray-filled node of the causal DAG 300, indicating that the number of medications taken per day by the patient is not a cause of the outcome 330 of the disease progression for that patient. In some aspects, the inclusion of non-causal variables and their corresponding nodes (e.g., such as the non-causal variable “# Of Medications Taken Per Day” and its corresponding node 302 in the causal DAG 300) in the estimation process(es) described herein may improve predictive performance while not introducing bias.

In another example, FIG. 3B depicts a causal DAG 350 representing the causal structure between the variables of FIG. 3A and a treatment effect 335 of the exposure 340 to the high quality radiology program, in accordance with some examples. The overall morbidity variable/node 312, the age variable/node 314, the social determinants of health variable/node 316, the number of medications taken per day variable/node 302, and the high quality radiology program exposure 340 are the same between causal DAG 300 of FIG. 3A and causal DAG 350 of FIG. 3B.

As noted above, causal DAG 350 is indicative of the causal structure between the variables under study and the treatment effect of the exposure (e.g., while causal DAG 300 of FIG. 3A indicates the causal structure between the different variables, the outcome, and the exposure). In the example of the causal DAG 350 of FIG. 3B, it can be seen that only the “Overall Morbidity” variable/node 312 causes changes in the treatment effect 335 (e.g., based on the treatment effect node 335 only being connected to the overall morbidity variable/node 312 in the causal DAG 350). Therefore, the “Overall Morbidity” variable/node 312 is the only variable that is a Direct Effect Modifier (DEM) of the CATE for the treatment effect 335 of the exposure 340 of a patient to the high quality radiology program.

In some aspects, one or more variables may be associated with the treatment effect 335 despite not causing any changes in the treatment effect 335. Such variables may be considered non-DEM (e.g., associative) effect modifiers of various types. For example, the “Age” variable/node 314 can be classified as an Indirect-type associative effect modifier. The “Body Part Imaged” variable/node 318b can be classified as a Common Cause-type associative effect modifier. The “Number of Medications Taken per Day” variable/node 302 can be classified as an associative effect modifier by Proxy. The SDOH variable/node 316 can be classified as an Indirect-type associative effect modifier. The “Sex” variable/node 322b is not an effect modifier of any type (e.g., and is not connected to any other variables/nodes in the causal DAG 350), and therefore is a variable that has no association with the treatment effect 335.

The “Treatment Effect” node 335 of the causal DAG 350 can correspond to a CATE estimation, which may be determined according to Eq. (1) and/or using a machine learning-based methodology for CATE estimation. In this example, the CATE may be represented as a function of the “Overall Morbidity” variable/node 312, which is the only variable identified as a DEM for the causal DAG 350, as described above. For example, the CATE may be given as:

$$CATE = - 0.2 + 0.15 * Overall Morbidity \qquad Eq . ( 2 )$$

Here, the expression-0.2+0.15*Average Overall Morbidity represents the Average Treatment Effect (ATE) of the “High Quality Radiology Program” exposure 340, determined over the observed population of patients under study or consideration. The ATE of receiving the High Quality Radiology Program exposure 340 is a 20% reduction in the probability of disease progression when Overall Morbidity 312 is a standardized variable with a mean of 0.

The coefficient of 0.15 is multiplied by the “Overall Morbidity” 312 for an individual patient, indicating that patients with higher Overall Morbidity 312 will benefit from the exposure 340 to the High Quality Radiology Program less than patients with relatively lower Overall Morbidity 312 (e.g., noting that benefit corresponds to a negative CATE value/reduction in probability of disease progression).

The remaining variables and nodes of the causal DAG 350 do not enter into the function for CATE, because these remaining variables do not directly impact the value of CATE and are not DEMs. In particular, “Age” 314, “Body Part Imaged” 318b, “Number Of Medications Taken Per Day” 302, and “Social Determinants of Health” 316 are all variables that are non-DEM (e.g., associative, rather than causal) effect modifiers, while the “Sex” 322b variable is not an effect modifier of any type and has no association with the treatment effect 335.

In some aspects, the covariates/matrix of pre-treatment variables X of Eq. (1) for a particular patient can include the corresponding values observed for each pre-treatment variable for that particular patient. Each patient's individual matrix of pre-treatment variables X can be provided as input to the CATE estimation and can be used to determine a corresponding CATE value for each patient. For example, consider the following two example patients:

| Patient | Variable | Value |
|---|---|---|
| Patient 1 | Age | 30 |
| | Sex | M |
| | Overall Morbidity | 0.3 |
| | Body Part Imaged | Chest |
| | #Of Medications | 4 |
| | SDOH | 2 |
| Patient 2 | Age | 40 |
| | Sex | M |
| | Overall Morbidity | 0.1 |
| | Body Part Imaged | Head |
| | #Of Medications | 0 |
| | SDOH | 2 |

The CATE for Patient 1=−0.2+0.15*0.3=−0.155.
The CATE for Patient 2=−0.2+0.15*0.1=−0.185.

Patient 2's probability of Disease Progression (e.g., the outcome 330 of causal DAG 300 of FIG. 3A) is reduced by a greater amount than Patient 1 from receiving the High Quality Radiology Program treatment/exposure 340 (e.g., 18.5% reduction for Patient 2 probability of Disease Progression outcome 330 given the exposure 340 to the high quality radiology program vs. 15.5% reduction for Patient 1 probability of Disease Progression outcome 330 given the same exposure 340 to the high quality radiology program), because Patient 2 has a lower Overall Morbidity 312 than Patient 1.

In some aspects, the matrix of pre-treatment variables X may include only those variables known to be effect modifiers that are at least associated with the estimated CATE. In this case, the “Sex” variable/node 322 would not be included in the matrix of covariates X, because the “Sex” variable 322 is not an effect modifier of any type. In other examples, the matrix of pre-treatment variables X can contain all observed pre-treatment variables, including both effect modifier variables and non-effect modifier variables. In such an example, the “Sex” variable 322 would be included in the matrix X.

More generally, it is contemplated that not all variables in the covariates/matrix of pre-treatment variables X provided as input to Eq. (1) and/or the CATE estimation of Eq. (2) need to be effect modifiers. The systems and techniques described herein for identification of DEMs are configured to work for a high-dimensional covariate space that can include a plurality of different variables where the underlying causal structure is not known. In some embodiments, after initially determining a CATE estimate, one or more correlation tests can be performed to reduce a search space for the presently disclosed Iterative Orthogonal Regression (IOR) technique (e.g., correlation tests are used to reduce the search space for the IOR technique, prior to performing the IOR). For example, before initiating the IOR, correlation tests can be used to reduce the search space to include only those variables that have significant associations with CATE (e.g., variables that are effect modifiers of some unknown type). The IOR can then be performed over the reduced search space of only variables with significant associations with CATE. In some examples, the presently disclosed IOR techniques can be performed to identify one or more DEMs for a CATE estimation using an assumption that all pre-treatment variables in X are conditioning variables for CATE. An assumption or requirement that all pre-treatment variables in X are effect modifiers of any type can be optional.

As mentioned previously, the term X can represent a matrix of covariates/pre-treatment variables for all patients (e.g., a respective value of each covariate/pre-treatment variable observed for each patient of the population under study). Each patient can have a unique vector of their individual covariates, x. Accordingly, returning to the CATE formulation of Eq. (1), (i.e., \hat{τ}(X)=E [Y(W)−Y(W*)|X]), it is noted that the CATE estimation function \hat{τ}(·) can operate over the covariate matrix X that represents or otherwise includes all the covariate values for all the patients in the population. Additionally, or alternatively, the CATE estimation function \hat{τ}(·) can operate over only the individual patient's unique vector of covariates, x, for each respective patient of the plurality of patients in the population.

In one illustrative example, the value of \hat{τ}(X) is a vector of CATE values each corresponding to a different patient of the population, where X is the covariate matrix representing all the covariate values for all the patients. In general, the CATE estimation function (·) is a function that maps covariate values to estimated conditional treatment effects. In some aspects, when used in this manner, CATE can also be understood to be representative or indicative of an Individual Treatment Effect (ITE), although it is noted that calculating CATE versus calculating ITE may utilize different respective assumptions with respect to the measurement of all effect modifiers. By comparison, the value of \hat{τ}(x) is a scalar value that represents the estimated CATE value for an individual patient, based on the patient's unique vector of covariates x. For instance, \hat{τ}(x) can be understood as the predicted average effect for a population where X=x, i.e., the average effect estimate conditional on x.

### Iterative Orthogonal Regression (IOR) for Direct Effect Modifier (DEM) Identification

Advances in causal machine learning have produced techniques for the non-parametric prediction of CATE, which may be seen to facilitate effect modifier identification through testing the association between variables and the estimated CATE. However, even with a perfect CATE estimation, machine learning-based techniques may be unable to differentiate between different types or degrees of effect modifiers for the CATE. For example, some machine learning-based techniques may be unable to differentiate between effect modifiers that cause effect heterogeneity (e.g., direct effect modifiers/DEMs, also referred to as causal effect modifiers) and effect modifiers that are simply associated with effect heterogeneity (e.g., associative effect modifiers/non-DEMs, such as indirect effect modifiers, common cause effect modifiers, and/or proxy effect modifiers).

In some applications or use cases of CATE, the distinction between different types of effect modifiers (e.g., DEMs vs. common cause, indirect, or proxy-type non-DEMs) may be unnecessary. For example, in a scenario where the goal is to use CATE estimation to predict which patients are most likely to benefit from a risky medical intervention (e.g., where receiving or not receiving the risky medical intervention is the exposure/binary treatment characterized by the CATE), learning associations between patient characteristics and treatment benefit can be sufficient, and there may not be a need to further differentiate the DEMs from non-DEMs in the set of learned associations. In other examples, there may be a need to go beyond merely identifying effect identifiers as the learned associations between patient characteristics and treatment benefit-such as in scenarios where the research goal is to better understand causal mechanisms, investigate policy bias, or optimize an existing program, etc. In such scenarios, the identification of DEMs may be required, and there is a need for the presently disclosed systems and techniques that can be used to identify one or more DEMs for an estimated CATE.

As used herein, effect modifier identification can be a separate, but related, task from direct effect modifier (e.g., DEM) identification. For instance, in the context of CATE estimation, the task of effect modifier identification can be performed to identify the subset of variables or covariates (e.g., from the CATE estimation) that are at least associated with effect heterogeneity. The task of direct effect modifier identification can be performed to further identify, from the previously identified effect modifiers, those effect modifiers that directly cause effect heterogeneity (e.g., DEMs). In other words, direct effect modifier identification can be understood as the task of differentiating DEMs from non-DEMs, given a set of effect modifiers of an initially unknown type.

In some existing approaches, effect modifiers are identified and tested through the inclusion of specific interaction terms in a fully parametric CATE model (e.g., where the specific interaction terms represent and/or are associated with the modeled effect modifiers). Such an approach is challenging to implement, and works only under a correct model specification and correct parametric assumptions. The correct model specification is a strong assumption, and in many cases is an unverifiable assumption. A challenge in the fully parametric approach to CATE estimation is that the true, underlying form of the relationship(s) between the outcome, treatment, and covariates is often unknown, and such an approach therefore risks model misspecification when the true relationships deviate from the assumed functional form used in the parametric model. For example, many real-world applications of causal inference and/or CATE estimation involve many variables where the underlying distributions, the relationships between variables, the exposure, and/or the outcome are unknown.

Causal machine learning techniques and models can be used to overcome some of the limitations described above of the fully parametric approach to CATE estimation. For example, one technique for identifying effect modifiers when manual model specification (e.g., manual input of the effect modifiers and/or specific interaction terms) is not feasible is to test for association between variables and estimated CATE. This approach can be effective for identifying effect modifiers in general (e.g., differentiating between variables or covariates that are at least associated with the CATE, versus variables or covariates that are not associated with the CATE). In particular, existing approaches to testing for association between variables and estimated CATE can be used to identify effect modifiers in general, but are unable and/or insufficient to differentiate DEMs from indirect, proxy, and common cause effect modifier types. This inability to differentiate DEMs from non-DEM effect modifier types is based at least in part on the fact that variables that do not directly modify treatment effects may still have significant associations with the estimated CATE (e.g., all DEMs are associated with the estimated CATE, but all variables associated with the estimated CATE are not DEMs).

Accordingly, the systems and techniques described herein can be used to differentiate between effect modifiers that cause effect heterogeneity (e.g., DEMs) and effect modifiers that are associated with but do not cause effect heterogeneity (e.g., indirect, proxy, and common cause effect modifier types). In one illustrative example, the systems and techniques can utilize Iterative Orthogonal Regression (IOR) for the identification of DEMs, as will be described in greater detail below.

### Step 1: Estimate CATE

In an initial step for performing IOR for the identification of DEMs, a CATE estimate can be generated, determined, or otherwise obtained. For example, a CATE estimate \hat{τ}(X) can be determined using Eq. (1), as described previously above. In some embodiments, CATE can be estimated using any applicable methodology for binary, categorical, or continuous treatments. In some aspects, the CATE estimate can be determined using one or more machine learning-based models, techniques, methodologies, etc.

### Step 2: Iterate Through Each Variable in X

By iterating through each variable in X (which is the matrix of pre-treatment variables/covariates for the population of patients), the IOR processing technique can be configured to generate a corresponding residualized version for each respective variable Xₚ in X. In one illustrative example, for each variable Xₚ, a model θₚ is fitted using all other variables in X (i.e., Xⱼ), and residuals are subsequently calculated. For instance, a residualized version \tilde{X}ₚ can be calculated for each variable Xₚ in X as follows:

$${θ}_{p} = E [ {X}_{p} \vert {X}_{j} ] \qquad Eq . ( 3 )$$

$${\tilde{X}}_{p} = {X}_{p} - {θ}_{p} \qquad Eq . ( 4 )$$

Notably, each residualized variable \tilde{X}ₚ is orthogonal (e.g., statistically independent) to each of the variables X, that were predictors in θₚ. The orthogonalization based on calculating a residualized version, \tilde{X}ₚ, of each variable Xₚ can be a key factor in de-confounding the relationship(s) between each variable Xₚ and the CATE \hat{τ}(X), which enables the subsequent identification of direct effect modifiers (DEMs) for the CATE. In various embodiments contemplated herein, the model θₚ used to generate the respective residualized version, \tilde{X}ₚ, of each variable Xₚ can be implemented without parametric or functional form assumptions, and various appropriate statistical and/or machine learning methods and models can be utilized for fitting θₚ.

In some aspects, the residualized \tilde{X}ₚ is a vector of values where each respective entry is the difference between the true value of Xₚ and the predicted value of Xₚ from the model θₚ of Step 2. For example, if Xₚ is the variable SDOH (e.g., SDOH variable/node 316 of FIGS. 3A and 3B, etc.), then the following relationship can be expressed:

$${θ}_{S D O H} = E [ SDOH \vert Age , Body Part Imaged , \\ Overall Morbidity , \# Medications ] \qquad Eq . ( 5 )$$

If modeling is subsequently performed using a main effects linear regression, the following can be obtained for the predicted SDOH (i.e., θ_{SDOH}):

$${θ}_{SDOH} = α + {β}_{Age} + {β}_{Body Part Imaged} + {β}_{Overall Morbidity} + {β}_{\# Medications} \qquad Eq . ( 6 )$$

Accordingly, the SDOH residuals (e.g., \tilde{X}_{SDOH}) can be calculated for each patient as:

$${\tilde{X}}_{SDOII} = {X}_{SDOII} - {θ}_{SDOII} = {X}_{SDOH} - ( α + {β}_{Age} + {β}_{Body Part Imaged} + {β}_{Overall Morbidity} + {β}_{\# Medications} ) \qquad Eq . ( 7 )$$

### Step 3: Iterate Through Each Variable in X and Estimate CATE Using the Respective Calculated Residualized Version \tilde{X}ₚ

In one illustrative example, CATE can be estimated using computed residuals according to:

$$\hat{τ} ( X ) = α + β {\tilde{X}}_{p} \qquad Eq . ( 8 )$$

For instance, the CATE, \hat{τ}(X), can be regressed on each residualized Xₚ (i.e., each \tilde{X}ₚ) using the equation above. After determining the respective residuals for the regression of each residualized Xₚ on the CATE, the set of residuals can be analyzed to determine the variables that are DEMs for the CATE. For example, each Xₚ whose residuals are determined to be significantly associated with CATE can be identified and labeled as a DEM. In some embodiments, p-values can be calculated for the residuals of each variable and used to indicate statistical significance for the identification of DEMs. Accordingly, parametric assumptions and form restrictions may be applied at this stage.

In one illustrative example, the residuals of Xₚ (i.e., the corresponding Xₚ) are regressed on the original \hat{τ}(X), i.e., the estimated CATE vector of Eq. (1) for the full covariate matrix X of the population. In the examples above, only the linear association between the CATE and the residualized variable \tilde{X}ₚ is tested. However, it is noted that in other embodiments, polynomial associations can additionally, or alternatively, be tested as well (e.g., by adding corresponding higher order predictors to the model specification for the regression of CATE on each residualized \tilde{X}ₚ). In some aspects, variables identified as DEMs can be the variables with residuals that are determined to be significantly associated with CATE (e.g., based on p-value, etc.). DEMs can be identified or otherwise determined with using a required or pre-determined p-value threshold for DEM identification. For example, while a particular p-value threshold may be used for a given DEM identification performed according to the steps above, the value of the p-value threshold can vary and/or can be user configured according to various parameters, preferences, etc. In some aspects, the p-value threshold corresponding to DEM identification of a variable can change between analyses performed according to the systems and techniques described herein for IOR.

In some aspects, standard and/or existing techniques for the identification of effect modifiers when manual model specification is not employed may fail to consider the causal structure of the variables, nor the reasons why a variable may be associated with CATE. Accordingly, the existing techniques may identify effect modifiers without differentiation among or between different types of effect modifiers. In some examples, the sub-categories of effect modifying variables can be understood and/or represented in the context of structuring the four types of effect modifying variables in a causal directed acyclic graph (DAG). The four types of effect modifying variables are DEMs, indirect, proxy, and common cause effect modifier types, which are described in turn below.

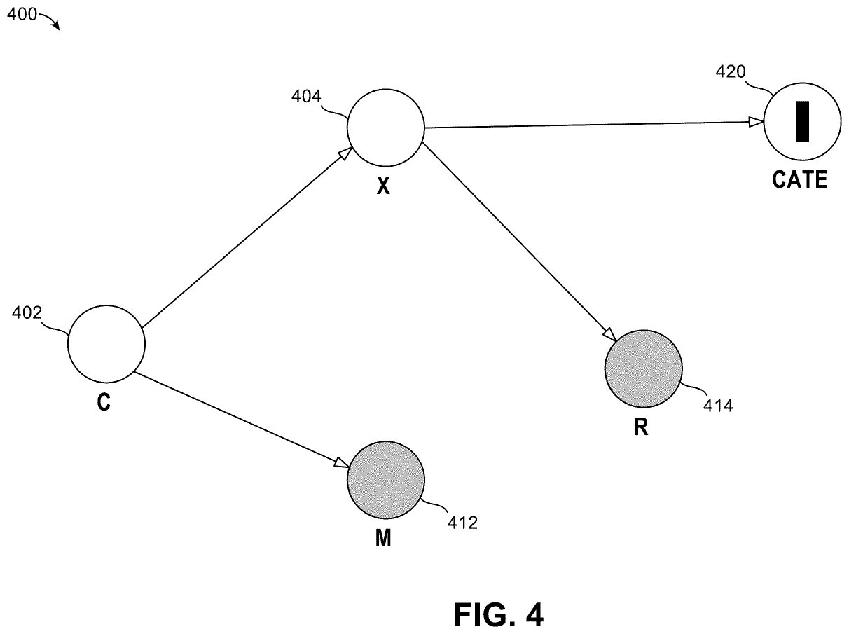

FIG. 4 is a diagram illustrating an example of a causal DAG 400 depicting different types of effect modifiers and their respective causal structures as they relate to a Conditional Average Treatment Effect (CATE), in accordance with some examples. For example, the causal DAG 400 illustrates the four types of effect modifiers and their respective causal structures as they relate to Conditional Average Treatment Effect (CATE).

In the example of causal DAG 400 and FIG. 4, a CATE node 420 corresponds to the Conditional Average Treatment Effect, which is directly affected by the variable/node X 404. This variable/node 404 represents that X is a direct (e.g., causal) effect modifier, and accordingly this variable X 404 directly modifies CATE 420.

The variable/node 402, labeled as C, is an indirect effect modifier-type variable. Node C 402 causes (e.g., has a causal relationship with/effect upon) the DEM node/variable X 404 and an additional node/variable M 412. Accordingly, node/variable C 402 is an indirect effect modifier because C 402 modifies CATE 420 indirectly through modifying X 404.

The variable/node M 412 is a common cause effect modifier-type variable, and is a non-DEM that is caused by the variable/node C 402. For instance, there exists a backdoor pathway M←C→X→CATE which induces a correlation between M 412 and CATE 420.

The variable/node R 414 is a proxy effect modifier-type variable, and is a non-DEM that is caused by X 404. For instance, there exists a backdoor pathway R←X→CATE which induces a correlation between R 414 and CATE 420.

Existing approaches for the identification of effect modifiers when manual model specification is not available can be seen as failing to consider the causal structure of the variables associated with the estimated CATE, as well as the reasons why a variable may be associated with CATE. For instance, CATE will be dependent on each of the four variable types above (e.g., effect modifier types X 404 (e.g., Direct), C 402 (e.g., Indirect), M 412 (e.g., Common Cause), and R 414 (e.g., Proxy). Accordingly, when applying existing techniques for effect modifier identification after performing machine learning-based CATE estimation, there will be no way to distinguish the DEMs from other effect modifier types unless strong assumptions are made regarding the underlying causal relationships as they relate to the effect as opposed to the outcome represented by the CATE estimation.

The four types of effect modifiers that can be associated with CATE are described in turn below with reference to corresponding example causal DAGs depicted in FIGS. 5-8. Table 1, below, summarizes the information and relationships represented in these causal DAGs:

TABLE 1
Example information and relationships represented in a causal DAG.
Reading Causal DAGs

| Component | Visual Depiction | Meaning |
|---|---|---|
| Node | White fill; | With Bar: Outcome |
| | Solid line | Without Bar: Variable that causes the outcome but not the |
| | | exposure |
| | White fill; | Confounding variable, a variable that causes both the |
| | Thick dashed line | exposure and the outcome (directly or indirectly) |
| | White fill; | With Triangle: The exposure of interest, the variable whose |
| | Thin dashed line | causal relationship with the outcome is of interest |
| | | Without Triangle: Variable that causes the exposure but not |
| | | the outcome |
| | Gray fill; | Causes neither the exposure nor the outcome |
| | Solid line | |
| Connecting | Any | The arrow indicates that the source variable/node causes the |
| Arrow | | terminating variable/node connected to the arrow |
| | Solid line | The causal pathway is not a direct effect path nor a biasing |
| | | pathway between the exposure and outcome |
| | Thick dotted line | The causal pathway is biasing the observed relationship |
| | | between the exposure and the outcome |
| | Thick dashed line | The causal pathway is a direct effect pathway between the |
| | | exposure and the outcome |

### Common Cause (M) Effect Modifier Type

FIG. 5 is an example of a causal DAG 500 corresponding to a Common Cause effect modifier variable/node M 512, where the common cause variable/node M 512 may be associated with—but does not directly modify—the treatment effect or cause effect heterogeneity, in accordance with some examples. Additionally, in the illustration of the causal DAG 500 for the Common Cause effect modifier variable/node M 512, a biasing pathway (shown as the thick dotted line arrows) exists between the common cause variable/node M 512 and the CATE 520.

Here, the CATE 520 includes a bar within the node 520, indicating that the CATE node 520 represents the outcome. The common cause, M, is shown as the node 512 having a dashed line circumference and enclosing a triangle (e.g., indicating node 512 is the exposure of interest, which is the variable whose causal relationship with the outcome CATE is of interest). An indirect effect modifier, C, is shown as the node 502, and represents a confounding variable that directly causes the exposure M 512 and indirectly causes the outcome CATE 520. A direct effect modifier, X, is shown as a node 504 without a bar, indicating that X is a variable that causes the outcome CATE 520 but not the exposure M 512. The variable R is depicted as a gray-filled node 514, indicating that variable R 514 causes neither the exposure nor the outcome.

As shown by dotted line connecting arrows in the causal DAG 500 of FIG. 5, there is a biasing pathway M←C→X→CATE that exists between the common cause M 512 and the CATE 520, which can be blocked by conditioning on C 502 or X 504. Residualizing M 512 by C 502, X 504, and R 514 results in no association between the residuals of M 512 (e.g., \tilde{M}) and CATE 520; therefore, M 512 is not identified or labeled as a DEM:

$${θ}_{M} = E [ M \vert C , X , R ] \qquad Eq . ( 9 )$$

$$\tilde{M} = M - {θ}_{M} \qquad Eq . ( 10 )$$

$$\tilde{M} ⊥ CATE \qquad Eq . ( 11 )$$

$$M CATE \qquad Eq . ( 12 )$$

$$M ⊥ CA T E \vert C , X , R \qquad Eq . ( 13 )$$

### Indirect (C) Effect Modifier Type

FIG. 6 is an example of a causal DAG 600 corresponding to an Indirect effect modifier variable/node C 602, where the Indirect effect modifier variable/node C 602 may be associated with—but does not directly modify—the treatment effect or cause effect heterogeneity.

Additionally, in the illustration of the causal DAG 600 for the Indirect effect modifier variable/node C 602, a biasing pathway (shown as the thick dotted line arrows) exists between the indirect effect variable/node C 602 and the CATE 620.

Here, the CATE 620 is again shown as solid node that includes a black vertical bar within, indicating that the CATE node 620 represents the outcome. In some embodiments, the CATE node 620 may be the same as or similar to the CATE node 520 of FIG. 5. The indirect effect modifier variable/node C is shown as the node 602 having a dashed line circumference and enclosing a triangle (e.g., indicating node 602 is the exposure of interest, which is the variable whose causal relationship with the outcome CATE is of interest).

As shown by the dotted line connecting arrows in the causal DAG 600 of FIG. 6, there is a biasing pathway C→X→CATE that exists between the indirect effect modifier C 602 and CATE 620, which can be blocked by adjusting for X 604. Residualizing C 602 by M 612, X 604, and R 614 results in no association between the residuals of the indirect effect modifier C 602 (e.g., C) and CATE 620; therefore, C 602 is not identified or labeled as a DEM:

$${θ}_{C} = E [ C \vert M , X , R ] \qquad Eq . ( 14 )$$

$$\tilde{C} = C - {θ}_{C} \qquad Eq . ( 15 )$$

$$\tilde{C} ⊥ CATE \qquad Eq . ( 16 )$$

$$C CATE \qquad Eq . ( 17 )$$

$$C ⊥ CATE \vert M , X , R \qquad Eq . ( 18 )$$

### Direct (X) Effect Modifier Type

FIG. 7 is an example of a causal DAG 700 corresponding to a Direct effect modifier (DEM) variable/node X 704 that directly modifies the treatment effect and/or can be seen to cause effect heterogeneity. In the causal DAG 700, no biasing pathway exists between X 704 and CATE 720. Instead, the pathway X→CATE is shown as a thick dashed arrow, indicating that the causal pathway from X 704 to CATE 720 is a direct effect pathway between the exposure and the outcome.

Residualizing X 704 by M 712, C 702, and R 714 still results in an association between the residuals of X 704 (e.g., X) and CATE 720. In other words, residualizing X 704 by the remaining variables M 712, C 702, and R 714 does not block or remove the association between X and CATE 720. Accordingly, X 704 is labeled as a DEM:

$${θ}_{X} = E [ X \vert M , C , R ] \qquad Eq . ( 19 )$$

$$\tilde{X} = X - {θ}_{X} \qquad Eq . ( 20 )$$

$$\tilde{X} CATE \qquad Eq . ( 21 )$$

$$X CATE \qquad Eq . ( 22 )$$

$$X CATE \vert M , C , R \qquad Eq . ( 23 )$$

### Proxy (R) Effect Modifier Type

FIG. 8 illustrates an example of a causal DAG 800 corresponding to an effect modifier variable/node R 814, where the node R 814 is an effect modifier by Proxy (also referred to as a proxy effect modifier, or proxy-type effect modifier). The proxy modifier R 814 is an effect modifier node/variable that may be associated with—but does not directly modify—the treatment effect or cause effect heterogeneity.

As illustrated by the thick dashed line arrows shown in the causal DAG 800 of FIG. 8, a biasing pathway R←X→CATE exists between the effect modifier by proxy (e.g., node/variable R 814) and the CATE 820. This biasing pathway can be blocked by adjusting for X 804. Residualizing R 814 by M 812, C 802, and X 804 results in no association between the residuals of R 814 (e.g., R) and the CATE 820; therefore, the node/variable R 814 is not identified or labeled as a DEM:

$${θ}_{R} = E [ R \vert M , C , X ] \qquad Eq . ( 24 )$$

$$\tilde{R} = R - {θ}_{R} \qquad Eq . ( 25 )$$

$$\tilde{R} ⊥ CATE \qquad Eq . ( 26 )$$

$$R CATE \qquad Eq . ( 27 )$$

$$R CATE \vert M , C , R \qquad Eq . ( 28 )$$

### Example Simulation Results

In one illustrative example, the presently disclosed systems and techniques for DEM identification using IOR were tested and validated using 250 data-generating processes of 2,500 samples, simulated where 20% of effect modifiers were DEMs. DEM status was then predicted using the p-value of the effect modifier's coefficient when individually regressing CATE. The presently disclosed IOR was applied and DEM status for each effect modifier was similarly re-predicted (e.g., according to steps 1-3 described previously above for IOR). The results demonstrate IOR's ability to identify DEMs with high precision and recall. The traditional/existing approach for DEM identification has high recall with low precision, indicating that the traditional/existing approach does not differentiate DEMs from general effect modifiers, even under perfect CATE estimation:

| Metric | Association with CATE | IOR |
|---|---|---|
| Precision | 0.27 | 0.87 |
| Recall | 1.00 | 0.99 |
| AUPRC | 0.34 | 0.90 |

In some aspects, the example simulation results above validate the disclosed systems and techniques for DEM identification using or otherwise based on IOR, indicating that IOR is a valid method for distinguishing between causal and associative effect modifiers under the examined settings of the simulation analysis. In some embodiments, IOR can be used to identify the direct effect modifiers of any exposure type, including binary, categorical, and/or continuous, etc. Direct effect modifier identification as described herein may be useful for various tasks such as uncovering causal mechanisms, researching health equity, program optimization, etc., among various others.

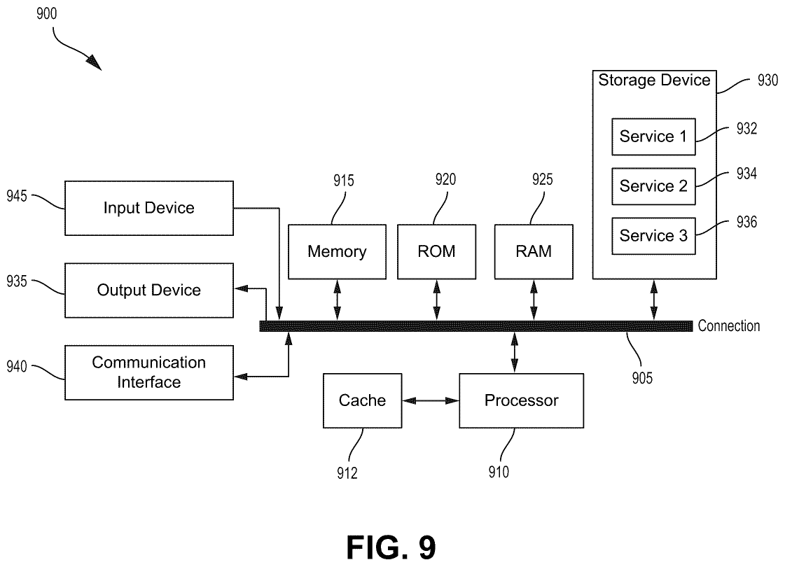

FIG. 9 illustrates an example computing device architecture 900 of an example computing device which can implement the various techniques described herein. In some examples, the computing device can include a mobile device, a wearable device, an XR device, a personal computer, a laptop computer, a video server, a video game console, a robotic device, a set-top box, a television, a camera, a server, or other device. The components of computing device architecture 900 are shown in electrical communication with each other using connection 905, such as a bus. The example computing device architecture 900 includes a processing unit (CPU or processor) 910 and computing device connection 905 that couples various computing device components including computing device memory 915, such as read only memory (ROM) 920 and random access memory (RAM) 925, to processor 910.

Computing device architecture 900 can include a cache of high-speed memory connected directly with, in close proximity to, or integrated as part of processor 910. Computing device architecture 900 can copy data from memory 915 and/or the storage device 930 to cache 912 for quick access by processor 910. In this way, the cache can provide a performance boost that avoids processor 910 delays while waiting for data. These and other modules can control or be configured to control processor 910 to perform various actions. Other computing device memory 915 may be available for use as well. Memory 915 can include multiple different types of memory with different performance characteristics. Processor 910 can include any general purpose processor and a hardware or software service, such as service 1 932, service 2 934, and service 3 936 stored in storage device 930, configured to control processor 910 as well as a special-purpose processor where software instructions are incorporated into the processor design. Processor 910 may be a self-contained system, containing multiple cores or processors, a bus, memory controller, cache, etc. A multi-core processor may be symmetric or asymmetric.

To enable user interaction with the computing device architecture 900, input device 945 can represent any number of input mechanisms, such as a microphone for speech, a touch-sensitive screen for gesture or graphical input, keyboard, mouse, motion input, speech and so forth. Output device 935 can also be one or more of a number of output mechanisms known to those of skill in the art, such as a display, projector, television, speaker device, etc. In some instances, multimodal computing devices can enable a user to provide multiple types of input to communicate with computing device architecture 900. Communication interface 940 can generally govern and manage the user input and computing device output. There is no restriction on operating on any particular hardware arrangement and therefore the basic features here may easily be substituted for improved hardware or firmware arrangements as they are developed.

Storage device 930 is a non-volatile memory and can be a hard disk or other types of computer readable media which can store data that are accessible by a computer, such as magnetic cassettes, flash memory cards, solid state memory devices, digital versatile disks, cartridges, random access memories (RAMs) 925, read only memory (ROM) 920, and hybrids thereof. Storage device 930 can include services 932, 934, 936 for controlling processor 910. Other hardware or software modules are contemplated. Storage device 930 can be connected to the computing device connection 905. In one aspect, a hardware module that performs a particular function can include the software component stored in a computer-readable medium in connection with the necessary hardware components, such as processor 910, connection 905, output device 935, and so forth, to carry out the function.

The term “device” is not limited to one or a specific number of physical objects (such as one smartphone, one controller, one processing system, and so on). As used herein, a device can include any electronic device with one or more parts that may implement at least some portions of this disclosure. While the description and examples use the term “device” to describe various aspects of this disclosure, the term “device” is not limited to a specific configuration, type, or number of objects. Additionally, the term “system” is not limited to multiple components or specific examples. For example, a system may be implemented on one or more printed circuit boards or other substrates, and may have movable or static components. While the description and examples use the term “system” to describe various aspects of this disclosure, the term “system” is not limited to a specific configuration, type, or number of objects.

Specific details are provided in the description to provide a thorough understanding of the aspects and examples provided herein. However, it will be understood by one of ordinary skill in the art that the aspects may be practiced without these specific details. For clarity of explanation, in some instances the present technology may be presented as including individual functional blocks including functional blocks comprising devices, device components, steps or routines in a method embodied in software, or combinations of hardware and software. Additional components may be used other than those shown in the figures and/or described herein. For example, circuits, systems, networks, processes, and other components may be shown as components in block diagram form in order not to obscure the aspects in unnecessary detail. In other instances, well-known circuits, processes, algorithms, structures, and techniques may be shown without unnecessary detail in order to avoid obscuring the examples.

Individual aspects and/or examples may be described above as a process or method which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general-purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as flash memory, memory or memory devices, magnetic or optical disks, flash memory, USB devices provided with non-volatile memory, networked storage devices, compact disk (CD) or digital versatile disk (DVD), any suitable combination thereof, among others. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

In some aspects, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Devices implementing processes and methods according to these disclosures can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Typical examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

In the foregoing description, aspects of the application are described with reference to specific examples thereof, but those skilled in the art will recognize that the application is not limited thereto. Thus, while illustrative examples of the application have been described in detail herein, it is to be understood that the inventive concepts may be otherwise variously embodied and employed, and that the appended claims are intended to be construed to include such variations, except as limited by the prior art. Various features and aspects of the above-described application may be used individually or jointly. Further, aspects of the present disclosure can be utilized in any number of environments and applications beyond those described herein without departing from the scope of the specification. The specification and drawings are, accordingly, to be regarded as illustrative rather than restrictive. For the purposes of illustration, methods were described in a particular order. It should be appreciated that in alternate examples, the methods may be performed in a different order than that described.

One of ordinary skill will appreciate that the less than (“<”) and greater than (“>”) symbols or terminology used herein can be replaced with less than or equal to (“≤”) and greater than or equal to (“≥”) symbols, respectively, without departing from the scope of this description.

Where components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” refers to any component that is physically connected to another component either directly or indirectly, and/or any component that is in communication with another component (e.g., connected to the other component over a wired or wireless connection, and/or other suitable communication interface) either directly or indirectly.

Claim language or other language reciting “at least one of” a set and/or “one or more” of a set indicates that one member of the set or multiple members of the set (in any combination) satisfy the claim. For example, claim language reciting “at least one of A and B” or “at least one of A or B” means A, B, or A and B. In another example, claim language reciting “at least one of A, B, and C” or “at least one of A, B, or C” means A, B, C, or A and B, or A and C, or B and C, or A and B and C. The language “at least one of” a set and/or “one or more” of a set does not limit the set to the items listed in the set. For example, claim language reciting “at least one of A and B” or “at least one of A or B” can mean A, B, or A and B, and can additionally include items not listed in the set of A and B.

The various illustrative logical blocks, modules, circuits, and algorithm steps described in connection with the examples disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random-access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration. Accordingly, the term “processor,” as used herein may refer to any of the foregoing structure, any combination of the foregoing structure, or any other structure or apparatus suitable for implementation of the techniques described herein.

What is claimed is:

1. A method for determining one or more direct effect modifiers for an exposure of interest, the method comprising:
determining a plurality of pre-treatment variables corresponding to characteristics of different subsets of an observed population for the exposure of interest, wherein the observed population comprises a plurality of individuals each having a respective exposure to the exposure of interest and a corresponding outcome;
generating a predicted conditional average treatment effect (CATE) information for the exposure of interest on the observed population, wherein the predicted CATE information is generated by a causal machine learning model configured with a covariate matrix of a set of variables comprising at least a portion of the plurality of pre-treatment variables;
for each respective variable included in the set of variables of the covariate matrix: fitting a configured model using a remaining set of variables with the respective variable removed, and determining a residual representation for the respective variable based on the fitting, wherein the residual representation for the respective variable is orthogonal to each variable in the remaining set of variables;
regressing the predicted CATE information on the residual representation for each respective variable to thereby determine additional residuals for each regressed residual representation;
determining association significance information between the CATE and the additional residuals of each respective variable; and
outputting an indication of the one or more direct effect modifiers (DEMs) for the exposure of interest, wherein the one or more direct effect modifiers are the respective variables of the covariate matrix having association significance above a threshold value with the CATE.

2. The method of claim 1, further comprising:
calculating p-values for the additional residuals of each respective variable with the CATE; and
identifying the one or more DEMs for the exposure of interest as the pre-treatment variables with calculated p-values above a configured threshold value of significance.

3. The method of claim 2, wherein the calculated p-values and the association significance information are the same.

4. The method of claim 1, wherein determining the association significance information between the CATE and the additional residuals of each respective variable comprises testing at least one of: linear association or polynomial associations between the CATE and the additional residuals.

5. The method of claim 1, wherein determining the residual representation for the respective variable based on the fitting of the model comprises a first step performed by an iterative orthogonal regression (IOR) engine, and wherein regressing the predicted CATE information comprises a second step performed by the IOR engine.

6. The method of claim 1, wherein the corresponding outcome for each individual of the observed population comprises a treatment effect of the individual's respective exposure to the exposure of interest.

7. The method of claim 1, wherein the one or more DEMs are identified from the plurality of pre-treatment variables as a respective one or more pre-treatment variables inferred to cause effect heterogeneity in the outcome for each individual's exposure to the exposure of interest.

8. The method of claim 7, further comprising outputting an indication of one or more pre-treatment variables identified from the plurality of pre-treatment variables as non-DEMs, wherein each non-DEM is associated with but does not cause the effect heterogeneity.

9. The method of claim 1, wherein the exposure comprises a binary exposure, a categorical exposure, or a continuous exposure.

10. An apparatus comprising:
at least one memory; and
at least one processor coupled to the at least one memory, the at least one processor configured to:
determine a plurality of pre-treatment variables corresponding to characteristics of different subsets of an observed population for an exposure of interest, wherein the observed population comprises a plurality of individuals each having a respective exposure to the exposure of interest and a corresponding outcome;
generate a predicted conditional average treatment effect (CATE) information for the exposure of interest on the observed population, wherein the predicted CATE information is generated by a causal machine learning model configured with a covariate matrix of a set of variables comprising at least a portion of the plurality of pre-treatment variables;
for each respective variable included in the set of variables of the covariate matrix: fit a configured model using a remaining set of variables with the respective variable removed, and determine a residual representation for the respective variable based on the fitting, wherein the residual representation for the respective variable is orthogonal to each variable in the remaining set of variables;
regress the predicted CATE information on the residual representation for each respective variable to thereby determine additional residuals for each regressed residual representation;
determine association significance information between the CATE and the additional residuals of each respective variable; and
output an indication of one or more direct effect modifiers (DEMs) for the exposure of interest, wherein the one or more direct effect modifiers are the respective variables of the covariate matrix having association significance above a threshold value with the CATE.

11. The apparatus of claim 10, wherein the at least one processor is further configured to:
calculate p-values for the additional residuals of each respective variable with the CATE; and
identify the one or more DEMs for the exposure of interest as the pre-treatment variables with calculated p-values above a configured threshold value of significance.

12. The apparatus of claim 11, wherein the calculated p-values and the association significance information are the same.

13. The apparatus of claim 10, wherein determining the association significance information between the CATE and the additional residuals of each respective variable comprises testing at least one of: linear association or polynomial associations between the CATE and the additional residuals.

14. The apparatus of claim 10, wherein determining the residual representation for the respective variable based on the fitting of the model comprises a first step performed by an iterative orthogonal regression (IOR) engine, and wherein regressing the predicted CATE information comprises a second step performed by the IOR engine.

15. The apparatus of claim 10, wherein the corresponding outcome for each individual of the observed population comprises a treatment effect of the individual's respective exposure to the exposure of interest.

16. The apparatus of claim 10, wherein the one or more DEMs are identified from the plurality of pre-treatment variables as a respective one or more pre-treatment variables inferred to cause effect heterogeneity in the outcome for each individual's exposure to the exposure of interest.

17. The apparatus of claim 16, wherein the at least one processor is further configured to output an indication of one or more pre-treatment variables identified from the plurality of pre-treatment variables as non-DEMs, wherein each non-DEM is associated with but does not cause the effect heterogeneity.

18. The apparatus of claim 10, wherein the exposure comprises a binary exposure, a categorical exposure, or a continuous exposure.

19. A non-transitory computer-readable storage medium having stored therein instructions which, when executed by a processor, cause the processor to perform operations comprising:
determining a plurality of pre-treatment variables corresponding to characteristics of different subsets of an observed population for an exposure of interest, wherein the observed population comprises a plurality of individuals each having a respective exposure to the exposure of interest and a corresponding outcome;
generating a predicted conditional average treatment effect (CATE) information for the exposure of interest on the observed population, wherein the predicted CATE information is generated by a causal machine learning model configured with a covariate matrix of a set of variables comprising at least a portion of the plurality of pre-treatment variables;
for each respective variable included in the set of variables of the covariate matrix: fitting a configured model using a remaining set of variables with the respective variable removed, and determining a residual representation for the respective variable based on the fitting, wherein the residual representation for the respective variable is orthogonal to each variable in the remaining set of variables;
regressing the predicted CATE information on the residual representation for each respective variable to thereby determine additional residuals for each regressed residual representation;
determining association significance information between the CATE and the additional residuals of each respective variable; and
outputting an indication of one or more direct effect modifiers (DEMs) for the exposure of interest, wherein the one or more direct effect modifiers are the respective variables of the covariate matrix having association significance above a threshold value with the CATE.

20. The non-transitory computer-readable storage medium of claim 19, wherein the instructions further cause the processor to perform operations comprising:
calculating p-values for the additional residuals of each respective variable with the CATE; and
identifying the one or more DEMs for the exposure of interest as the pre-treatment variables with calculated p-values above a configured threshold value of significance.
